(19) United States
(12) Patent Application Publication
(10) Pub. No.: US 2019/0358249 A1
(43) Pub. Date: Nov. 28, 2019

(54) Injectable Combination Products Of Fosaprepitant And 5-HT3 Blocker

(71) Applicant: Navinta III Inc, Boca Raton, FL (US)

(72) Inventor: Sandipkumar Arvindbhai Patel, Lawrenceville, NJ (US)

(21) Appl. No.: 16/418,496

(22) Filed: May 21, 2019

Related U.S. Application Data
(60) Provisional application No. 62/674,899, filed on May 22, 2018.

(51) Int. Cl. A61K 31/675; A61K 31/473; A61K 31/4178; A61K 9/00; A61K 47/26; A61K 47/02

(52) U.S. Cl. CPC A61K 31/675; A61K 31/473; A61K 31/4178; A61K 9/19; A61K 47/26; A61K 47/02; A61K 9/0019

(57) ABSTRACT

Sterile pharmaceutical products of Fosaprepitant and a 5-HT3 blocker for parenteral administration including a container containing a formulation of Fosaprepitant or a pharmaceutically acceptable salt thereof and 5-HT3 blocker. The formulations may comprise surface-active agent and a pharmaceutically acceptable vehicle. The formulations may be lyophilized and diluted prior to administration or ready to use liquids or a pre-lyophilization solution.

### FIELD OF THE INVENTION

The present invention relates to an injectable combination pharmaceutical product comprising Fosaprepitant or a suitable salt thereof and a 5-HT3 blocker, preferably Ondansetron or a suitable salt thereof, in a single dosage form.

### BACKGROUND OF THE INVENTION

Nausea and vomiting, which often follows chemotherapy, is a severe and distressing side effect of many chemotherapeutics. Current treatment of these effects includes nurokinin-1 (NK1) receptor antagonists and 5-hydroxytryptamine 3 (5-HT3) receptor antagonists to a subject in need thereof. With the intent of developing more effective treatments for chemotherapy induced nausea and vomiting (CINV), studies were carried out to demonstrate the efficacy of combining the NK-1 receptor antagonist Aprepitant with a 5-HT3 receptor antagonist (ondansetron) and a corticosteroid (dexamethasone). Results showed that addition of Aprepitant to a standard treatment regimen of ondansetron and dexamethasone was generally well-tolerated and provided consistently superior protection against CINV in subjects receiving highly emetogenic cisplatin-based chemotherapy (Hesketh et al., 2003, J Clin Oncol. 21:4112-4119).

Consequently, combination products of 5-HT3 blocker and NK1 receptor agonist have been proposed. For example, US Patent Application Publication Nos. 20150320866 and 20170232107 disclose sustained or controlled release of 5-HT3 receptor antagonist and/or NK-1 receptor antagonist, which may be selected from ondansetron and Fosaprepitant, respectively, in a semi-solid delivery vehicle. Particular embodiments disclosed include Aprepitant or Fosaprepitant and granisetron in polyorthoester excipient for subcutaneous injection.

Fosaprepitant has good solubility in water, while its pharmaceutically active degradant Aprepitant has minimal solubility. Therefore, for certain formulations, it is desirable to use Fosaprepitant as the active agent. However, a major stability issue with Fosaprepitant is its hydrolysis in aqueous media to form the parent Aprepitant, which is practically insoluble in water and generates particles in the injectable solution, which is undesirable. To avoid the degradation of Fosaprepitant to Aprepitant in aqueous media, Fosaprepitant is lyophilized to remove water. Scientific discussion of EMA for such lyophilized Fosaprepitant product describes that “The process has been optimized to prevent degradation of Fosaprepitant during manufacture especially with regard to temperature, pH, and water content.”¹ ¹ http://www.ema.europa.eu/docs/en_GB/document_library/EPAR_-_Assessment_Report_-_Variation/human/000743/WC500097052.pdf, at page 7, sec. 2.2.3.1, para 8.

U.S. Pat. No. 9,913,853 suggests that it may be desirable to use ready-to-use liquid formulation of Fosaprepitant in conjunction with another pharmacologically active agent, which may include corticosteroids, 5-HT3 receptor antagonists or chemotherapeutic agent. However, no combination formulations in which Fosaprepitant is formulated in a single dosage form with another active agent are disclosed.

AKYNZEO® is a combination drug product of Fosnetupitant and Palonosetron hydrochloride recently approved by United States Food and Drug Administration. AKYNZEO® (235 mg fosnetupitant/0.25 mg palonosetron) for injection is a combination product of fosnetupitant, a prodrug of netupitant, which is a substance P/neurokinin 1 (NK-1) receptor antagonist, and palonosetron hydrochloride, a serotonin-3 (5-HT3) receptor antagonist. AKYNZEO® for injection is available for intravenous infusion and is supplied as a sterile lyophilized powder in a single-dose vial. Each vial contains 235 mg of fosnetupitant (equivalent to 260 mg fosnetupitant chloride hydrochloride) and 0.25 mg of palonosetron (equivalent to 0.28 mg of palonosetron hydrochloride). The inactive ingredients are edetate disodium (6.4 mg), mannitol (760 mg), sodium hydroxide and/or hydrochloric acid (for pH adjustment).² ² https://www.accessdata.fda.gov/drugsatfda_docs/label/2018/210493s0001bl.pdf, at page 12-13, sec. 11.

Although, in practice, Fosaprepitant and Ondansetron are used together, a combination dosage form of Fosaprepitant and Ondansetron is not currently available in the market. Currently, for administration by intravenous injection, both of these products are available as separate products. EMEND (Fosaprepitant Dimeglumine for Injection) is a lyophilized product and ZOFRAN (Ondansetron HCl Injection) is a liquid product.

EMEND is used by reconstitution and further dilution before administration to patient. The indication of EMEND is prevention of nausea and vomiting associated with highly emetogenic cancer chemotherapy and moderately emetogenic cancer chemotherapy. A currently available product of Fosaprepitant Dimeglumine for Injection 150 mg/vial is a lyophilized powder that is stored at 2-8° C. The product should be reconstituted using 5 mL 0.9% Sodium chloride Injection and further diluted to 150 mL using 0.9% Sodium Chloride Injection. The final diluted solution must be stored at or below 25° C. (or room temperature) and used in 24 hours. In-house analysis of EMEND (lyophilized sample marketed in US) shows pH 8.5.

ZOFRAN is used by dilution before administration to a patient. The indication of ZOFRAN is also for highly emetogenic cancer chemotherapy and moderately emetogenic cancer chemotherapy. Currently available Ondansetron hydrochloride Injection dosages are 2 mg/mL, in 2 mL or 20 mL (as multiple dose vial). The recommended adult intravenous dosage of ondansetron hydrochloride is three 0.15 mg/kg doses up to a maximum of 16 mg per dose. The first dose is infused over 15 minutes beginning 30 minutes before the start of emetogenic chemotherapy. Subsequent doses (0.15 mg/kg up to a maximum of 16 mg per dose) are administered 4 and 8 hours after the first dose of ondansetron hydrochloride. The pH of the injection solution is 3.3 to 4.0.³ ³ https://www.accessdata.fda.gov/drugsatfda_docs/label/2017/020007s0471bl.pdf, at Page 13, section 11: Description.

In recent years, the ZOFRAN brand product has been discontinued from the market. The Orange Book indicates that the product was not withdrawn for safety or efficacy reasons. There are still a number of ANDAs approved for generic Ondansetron HCl injection formulations and generic products are currently commercially distributed and utilized by patients.

Ondansetron solubility in water is 3.2% and 0.8% in saline. The pH of a 1% w/v solution in water is about 4.6. The pKa is 7.4 such that free base precipitates when the pH is above the range 5.7-7 (Ref. par 3, page 6).⁴ Ondansetron hydrochloride is more stable in acidic media than at neutral pH.⁵ ⁴ http://www.mhra.gov.uk/home/groups/par/documents/websiteresources/con2023944.pdf⁵ http://www.mhra.gov.uk/home/groups/par/documents/websiteresources/con2023944.pdf. at par 3, page 6.

Based on the generally available information, it is evident that Ondansetron HCl has more solubility and stability at lower pH, while Fosaprepitant has more stability at higher pH, which makes formulation of a stable combination liquid product difficult.

There is a desire to treat CINV with a combination of NK1 and 5-HT3 antagonists. There are many therapeutic benefits to the patient and the provider to combine NK1 and 5-HT3 antagonists into a single dosage form for administration to patients in need thereof.

In particular, there is a desire to have a combination formulation of Fosaprepitant and 5-HT3 blocker. Namely, there is a desire to have stable injectable combination formulations of Fosaprepitant and Ondansetron.

ALOXI® (Palonosetron Hydrochloride) Injection is supplied ready for intravenous administration at a concentration of 0.05 mg/mL (50 mcg/mL). The dosage of ALOXI® for adults is 0.25 mg infusion over 30 seconds beginning approx. 30 min before the start of chemotherapy. Palonosetron Hydrochloride is freely soluble in water.⁶ Furthermore, it is very soluble in aqueous media across the physiological pH range.⁷ ⁶ https://www.accessdata.fda.gov/drugsatfda_docs/label/2014/021372s0201bl.pdf at Page 2 section 2.⁷ http://www.ema.europa.eu/docs/en_GB/document_library/EPAR_-_Public_assessment_report/human/004069/WC500205049.pdf at Page 7, section 2.2.2

AKYNZEO® (235 mg fosnetupitant/0.25 mg palonosetron) for injection is a lyophilized product, which needs following steps before administration (i) Aseptically inject 20 mL 5% Dextrose Injection, USP or 0.9% Sodium Chloride Injection, USP into the vial. Ensure the solvent is added to the vial along the vial wall and not jetted in order to prevent foaming. Swirl the vial gently. (ii) Aseptically prepare an infusion vial or bag filled with 30 mL of 5% Dextrose Injection, USP or 0.9% Sodium Chloride Injection, USP (iii) Aseptically withdraw the entire volume of reconstituted solution from the AKYNZEO® vial and transfer it into the infusion vial or bag containing 30 mL of 5% Dextrose Injection, USP or 0.9% Sodium Chloride Injection, USP to yield a total volume of 50 mL. (iv) Gently invert the vial or bag till complete dissolution. (v) Before administration, inspect the final diluted solution for particulate matter and discoloration. Discard the vial or bag if particulates and/or discoloration are observed. Based on this information from the product insert, it is evident that strict aseptic processing is required for reconstitution and further dilution of AKYNZEO® to avoid microbial contamination.⁸ ⁸ https://www.accessdata.fda.gov/drugsatfda_docs/label/2018/210493s0001bl.pdf at Page 3, section 2.2

Based on the product information of ALOXI® and AKYNZEO®, there is a need for a ready-to-use combination injectable product of 5-HT3 blocker, such as Palonosetron, and NK1 blocker, such as Fosaprepitant.

### SUMMARY OF THE INVENTION

The present invention is intended as combination injectable product of Fosaprepitant and 5-HT3 blocker in a single dose container. This dosage form is liquid or in lyophilized form. The proposed formulation may be supplied in one container for use as a ready to use liquid product or lyophilized product to further reconstitute and/or dilute.

In certain embodiments, the container is a glass vial.

In one aspect, the invention comprises a liquid sterile pharmaceutical product of Fosaprepitant and a 5-HT3 blocker for parenteral administration including a container containing a liquid formulation of Fosaprepitant or a pharmaceutically acceptable salt thereof, 5-HT3 blocker, surface-active agent at about 0.01% to 20% w/v, and a pharmaceutically acceptable vehicle. In some of those embodiments, the product is sterile according to USP 41 <71>.

In some embodiments, the surface-active agent is 0.05% to 15% w/v of the formulation.

In certain embodiments, the surface-active agent is Polysorbate 80.

In some embodiments, the product further comprises at least one of a tonicity agent, pH adjusting agent and chelating agent.

In certain embodiments, the 5-HT3 blocker is selected from the group consisting of Ondansetron, Palonosetron, Granisetron, Dolesetron, Tropisetron, and Zatisetron.

In some preferred embodiments, the formulation comprises Fosaprepitant Dimeglumine and Palonosetron Hydrochloride. In some of those embodiments, the concentration of Fosaprepitant Dimeglumine is from 0.5 mg/mL to 250 mg/mL and the concentration of Palonosetron Hydrochloride is from 1 μg/mL to 500 μg/mL.

In another aspect, the invention comprises lyophilized sterile pharmaceutical product of Fosaprepitant and 5-HT3 blocker for parenteral administration having a container containing a lyophilized powder of Fosaprepitant or a pharmaceutically acceptable salt thereof; and a 5-HT3 blocker.

In certain embodiments, the 5-HT3 blocker of the lyophilized product is selected from the group consisting of Ondansetron, Palonosetron, Granisetron, Dolesetron, Tropisetron, and Zatisetron. In some of those embodiments, the 5-HT3 blocker is Ondansetron or a salt thereof. In certain particularly preferred embodiments, the Ondansetron or a salt thereof is Ondansetron Hydrochloride. In other embodiments, the 5-HT3 blocker is Palonosetron or a salt thereof. In certain particularly preferred embodiments, the Palonosetron or a salt thereof is Palonosetron Hydrochloride.

In some embodiments, the lyophilized powder is reconstituted with a diluent to contain Fosaprepitant Dimeglumine from about 5 mg/mL to 150 mg/mL and Ondansetron HCl from about 0.5 mg/mL to 32 mg/mL for parenteral administration. In some of those embodiments, the diluent for reconstitution is water for injection, 0.9% sodium chloride injection, 5% Dextrose, a tonicity adjusting agent or any combination thereof. In some embodiments, the reconstituted solution is further diluted at least 5 times before parenteral administration.

In yet another aspect, the invention comprises a pre-lyophilization solution of Fosaprepitant Dimeglumine and Ondansetron Hydrochloride including a solution containing Fosaprepitant Dimeglumine from about 0.5 mg/mL to 250 mg/mL and Ondansetron Hydrochloride from about 0.5 mg/mL to 32 mg/mL.

In a further aspect, the invention comprises a pre-lyophilization solution of Fosaprepitant Dimeglumine and Palonosetron Hydrochloride including a solution containing Fosaprepitant Dimeglumine from about 0.5 mg/mL to 250 mg/mL and Palonosetron Hydrochloride from about 0.01 mg/mL to 0.5 mg/mL.

In certain embodiments, the pre-lyophilization solutions contain water, alcohol or a combination thereof as solvent.

In some embodiments the pre-lyophilization solutions contain aqueous solvent or non-aqueous solvent or a combination thereof.

### DESCRIPTION OF THE DRAWINGS

FIG. 1 shows a micellar formulation of Fosaprepitant Dimeglumine with Polysorbate 80 surfactant.

FIG. 2 shows interaction of Aprepitant with Polysorbate 80 surfactant.

### DETAILED DESCRIPTION OF THE INVENTION

The goal of antiemetic therapy is the complete prevention of CINV. Currently, there are two major categories of drugs with the highest therapeutic index for the management of CINV. These are: 5-HT3 receptor antagonists (e.g., granisetron and ondansetron) and NK1 receptor antagonists (e.g., Aprepitant and Fosaprepitant). Unfortunately, as treatment with any single agent has not been shown to provide complete prevention of CINV for all or almost all patients in need, studies are in progress to identify combinations which can provide the desired efficacy. While it is desirable to provide prophylactic treatment to prevent any onset of nausea and/or vomiting, the present disclosure is understood to also include treatment after onset of symptoms such as nausea and/or vomiting.

Provided herein are pharmaceutical compositions comprising a 5-HT3 receptor antagonist and a NK-1 receptor antagonist to provide combination therapy for CINV (chemotherapy induced nausea and vomiting), RINV (radiotherapy induced nausea and vomiting), and other related diseases or disorders. In a preferred embodiment, a delivery vehicle is formulated which, when administered parenterally, can provide simultaneous delivery of the active agents to a subject in need thereof. The vehicle contains both the 5-HT3 receptor antagonist and the NK-1 receptor antagonist, wherein the formulation provides immediate release of both active agents upon administration.

As used herein, the term “pharmaceutically acceptable” means that which is useful in preparing a pharmaceutical composition that is generally safe and non-toxic.

The term “parenteral” or “injectable” refers to routes selected from subcutaneous (SC), intravenous (IV), intramuscular (IM), intradermal (ID), intraperitoneal (IP) and the like.

Suitable 5-HT3 antagonists include, but are not limited to, metoclopramide, ondansetron, granisetron, tropisetron, palonosetron, and dolasetron, including all pharmaceutically acceptable salts thereof. Currently marketed anti-emetics which have 5-HT3 receptor antagonists include SANCUSO (granisetron hydrochloride) and ZOFRAN ODT (ondansetron), ALOXI® (palonosetron hydrochloride), ANZEMET (dolasetron mesylate), NAVOBAN (tropisetron), and IRIBO (ramosetron).

In certain embodiments, the 5-HT3 antagonist may be selected from the group consisting of Ondansetron, Palonosetron, Granisetron, Dolesetron, Tropisetron, and Zatisetron. The 5-HT3 blocker is preferably Ondansetron and/or Palonosetron.

The therapeutic agent may also be used in the form of one or more salts or mixtures of the agent in its unmodified form and in salt form. Suitable pharmaceutically acceptable salts include acid addition salts which may, for example, be formed by mixing a solution of the compound with a solution of a pharmaceutically acceptable acid such as hydrochloric acid, fumaric acid, maleic acid, succinic acid, acetic acid, citric acid, tartaric acid, carbonic acid, phosphoric acid, sulfuric acid and the like. Salts of amine groups may also comprise the quaternary ammonium salts in which the amino nitrogen atom carries an alkyl, alkenyl, alkynyl or aralkyl group. Where the compound carries an acidic group, for example a carboxylic acid group, the present disclosure also contemplates salts thereof, preferably non-toxic pharmaceutically acceptable salts thereof, such as the sodium, potassium and calcium salts thereof.

The concentration of the 5-HT3 receptor antagonist in the lyophilized composition may vary from about 0.01 wt % to 10 wt %, 0.1 wt % to 10 wt %, 1 wt % to 10 wt %, 2 wt % to 8 wt %, 2 wt % to 5 wt %, 2 wt % to 3 wt %, or 1 wt % to 5 wt %. The preferred amount used in product depends on type and potency of 5-HT3 blocker.

As used herein, the term “about” is defined as ±10%, preferably ±5%.

Suitable NK-1 receptor antagonists for use in the presently described pharmaceutical compositions in combination with one or more 5-HT3 receptor antagonists include RP 67580 ((3aR,7aR)-Octahydro-2-[1-imino-2-(2-methoxyphenyl)ethyl]-7,7-diphenyl-4H-isoindol)), WIN 51078 (17-.beta.-Hydroxy-17-.alpha.-ethynyl-5-.alpha.-androstano[3,2-b]pyrimido-[1,2-a]benzimidazole), 1-733,060, (((2S,3S)-3-[[3,5-bis(Trifluoromethyl)phenyl]nethoxy]-2-phenylpiperidine hydrochloride), I-703,606 (cis-2-(Diphenylmethyl)-N-([2-iodophenyl]methyl)-1-azabicyclo(2.2.2)octan-3-amine) MDL 105,212 (R)-1-[2-[3-(3,4-dichlorophenyl)-1-(3,4,5-trimethoxybenzoyl)-pyrrolidin-3--yl]-ethyl]-4-phenylpiperidine-4-carboxamide hydrochloride), serlopitant, maropitant, Antagonist D, Aprepitant, Fosaprepitant, R116301, CGP49823, CP-96345, CP-99994, GR-203040, MDL-103392, 1-760735, SDZ-NKT-343, nolpitanitium (SR-140333), AV608, rolapitant, SCH 900978, AV608, GSK424887 (GlaxoSmithKline), GSK206136 (GlaxoSmithKline), GR-205171, CP-99994, TAK 637 ((S)-7-(3,5-Bis-trifluoromethyl-benzyl)-9-methyl-5-p-tolyl-8,9,10,11-tetr-ahydro-7H-1,7,11a-triaza-cycloocta[b]naphthalene-6,12-dione), LY303870 ([(R)-1-[N-(2-methoxybenzyl)acetylamino]-3-(1H-indol-3-yl)-2-[N-(2-(4-(pi-peridin-1-yl)piperidin-1-yl)acetyl)amino]propane]), LY686017 ((2-chloro-phenyl)-{2-[5-pyridin-4-yl-1-(3,5-bistrifluoromethyl-benzyl)-1-H-[1,2,3]triazol-4-yl]-pyridin-3-yl}-methanone), E-6006, casopitant/GW679769 ((2R,4S)-4-(4-acetylpiperazin-1-yl)-N-[(1R)-[3,5-bis(trifluoromethyl)phen-ypethyl]-2-(4-fluoro-2-methylphenyl)-N-methylpiperidine-1-carboxamide), vestipitant, orvepitant and orvepitant maleate, netupitant, ezlopitant, CP-122721, MPC-4505 (Myriad Genetics, Inc.), CP-122721 (Pfizer, Inc.), CJ-1 2,255 (Pfizer, Inc.), SRR 240600 (Sanofi-Aventis), or TA-5538 (Tanabe Seiyaku Co.) including all pharmaceutically acceptable salts thereof.

The NK-1 receptor antagonist is preferably Fosaprepitant or a pharmaceutically acceptable salt thereof, most preferably, Fosaprepitant Dimeglumine.

The concentration of the NK-1 receptor antagonist, preferably Fosaprepitant or a pharmaceutically acceptable salt thereof, in the lyophilized composition may vary from about 0.5 wt % to 80 wt %, 0.5 wt % to 35 wt %, 0.5 wt % to 30 wt %, 0.5 wt % to 20 wt %, 0.5 wt % to 10 wt %, 5 wt % to 40 wt %, 10 wt % to 35 wt %.

The formulation may be provided as a single unit lyophilized sterile pharmaceutical formulation of Fosaprepitant Dimeglumine and 5-HT3 blocker for parenteral administration in a container. In certain embodiments, the compositions of the present invention can be formulated as a “single use dosage” or “single unit formulation” which refers to a premixed composition that is disposed within a sealed container or vessel as a one dose per container or vessel formulation.

As used herein, the term “container” refers to materials that are in direct contact with the active ingredients and are suitable as pharmaceutical packaging components (e.g. bottles, vials, closures). Guidelines on suitable packaging for pharmaceutical products are published by e.g. the World Health Organization and are well known to those of skill in the art. In preferred embodiments, the container is a glass vial.

As used herein to describe the formulations and products of the present invention, the term “sterile” means a product that has been filter sterilized. In certain embodiments, the sterilizing filter is a 0.22μ or 0.45μ filter.

In preferred embodiments, the parenteral formulation is injected intravenously. As a result, in particular preferred embodiments, the formulation can be labeled as sterile according to the criteria of sterility in the United States Pharmacopoeia 41 <71>, “41 USP”. Further regulations for sterility of the final product include the European Pharmacopoeia (Ph. Eur. section 2.6.1), and the Japanese Pharmacopoeia (JP section 54). These methods have been harmonized with the USP methods and results generated under these sources can be considered equivalent to testing conducted according to USP <71>. Preferably, therapeutically acceptable products have been produced by a method which provides assurance of sterility according to the US Pharmacopoeia 40<71> or USP 41<71>.

In certain preferred embodiments, the single unit sterile pharmaceutical formulation is comprised of Fosaprepitant Dimeglumine and Ondansetron Hydrochloride for parenteral administration. Most preferably, the single unit sterile pharmaceutical formulation comprises lyophilized Fosaprepitant Dimeglumine and Ondansetron Hydrochloride and is suitable for parenteral administration.

Consequently, the invention also comprises a liquid solution for lyophilization containing Fosaprepitant Dimeglumine from about 5 mg/mL to about 150 mg/mL. The liquid solution for lyophilization may further comprise Ondansetron HCl from about 0.5 mg/mL to about 32 mg/mL.

The liquid solution may further contain water, alcohol or combinations thereof as solvent. The liquid solution can comprise aqueous solvent or non-aqueous solvent or combinations thereof.

The formulations preferably further comprise a surface active agent. In certain preferable embodiments, the formulations comprise Polysorbate 80.

The surface-active agent may be present at about 5% to 30% w/w of the lyophilized formulation, preferably about 10% to 25% w/w. In some embodiments, the surface active agent is present from about 10% to about 20% w/w. In other embodiments, the surface-active agent is present from about 10% to about 15% w/w, preferably 10% to 12% w/w. The concentration of surfactant is dependent on the amount of Fosaprepitant active ingredient, with higher concentrations of active requiring greater amounts of surfactant.

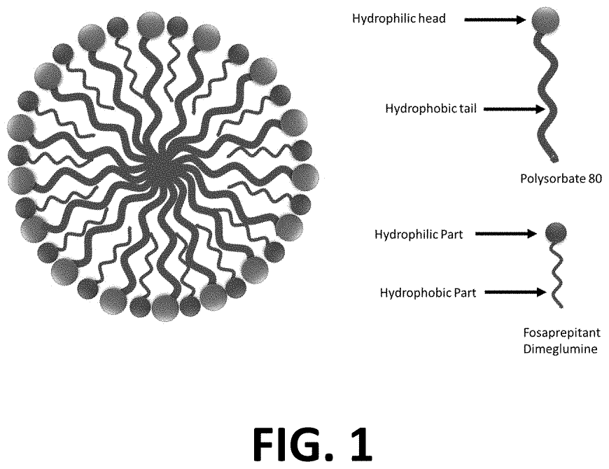

Without wishing to be bound, it is theorized that surface-active agent, such as Polysorbate 80, a non-ionic surfactant, may form mixed micelles. Fosaprepitant may become entrapped in the micelles and possibly remain protected from outer degrading environment as presented in FIG. 1.

Dissolving Fosaprepitant at room temperature to get a clear solution followed by addition of Polysorbate 80 may possibly attract the non-polar part of Fosaprepitant at non-polar side chain (tail) of Polysorbate 80. As soon as Polysorbate 80 concentration increases during dissolution, it starts forming micelles above CMC concentration and Fosaprepitant starts to be entrapped into micelles. After complete dissolution of Polysorbate 80, and further reduction of temperature from room temperature to 2-8° C., the solubility of Polysorbate 80 free form (other than micellar form) will be reduced and more micelle will be formed to capture more amounts of Fosaprepitant in micelles.

In another aspect, the formulation is possibly a mixed micellar formulation of Fosaprepitant Dimeglumine and Polysorbate 80. Formation of mixed micelles with surfactant provides stability of the solution due to protection of labile group in the inner core of micelles. We have found that liquid formulation of Fosaprepitant Dimeglumine has about 0.4% Aprepitant (main degradation product of Fosaprepitant) after 90 days at 2-8° C.

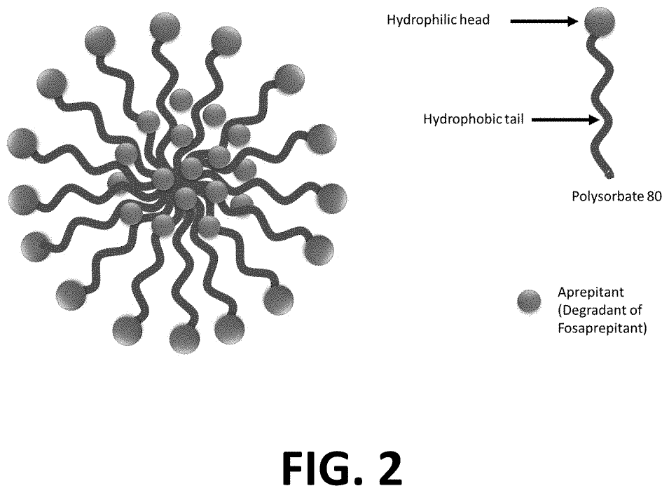

In another aspect, the proposed formulation is possibly mixed micellar formulation of Fosaprepitant Dimeglumine with Polysorbate 80 and Aprepitant (degradant of Fosaprepitant) with Polysorbate 80. Fosaprepitant degrades to its therapeutically active metabolite Aprepitant, which has very low solubility in water and thus forms visible and subvisible particles during storage. Entrapment of Aprepitant in micelles of Polysorbate 80 (as depicted in FIG. 2) during the storage of Fosaprepitant containing liquid formulation, increases the stability of product by avoiding generation of particulate matter.

In another aspect of the invention, the order of addition of ingredients, initial concentration and temperature of solution may possibly stabilize the formulation in liquid dosage form. Addition of surface-active agent after dissolving Fosaprepitant may possibly lead to more efficient entrapment. Cooling of concentrated bulk solution may possibly increase the degree of micelle formation or interaction. Holding of solution at specific temperature range may possibly equilibrate the micelles rapidly.

Fosaprepitant Dimeglumine combination liquid dosage forms can be prepared by dissolving Fosaprepitant or its pharmaceutically acceptable salt thereof along with 5-HT3 blocker in part of water, adding surface-active agent and further adding water to make the volume up to batch size.

The invention is not limited to the above proposed mechanisms. The proposed stabilization mechanisms may not be the only operative mechanisms, there could be other unknown interactions that could stabilize the dosage form.

A bulking agent may be present in formulation at about 5% to 60% w/w, preferably about 30% to 50% w/w. The bulking agent described herein is selected from the group consisting of but not limited to Lactose, Mannitol, Dextrose, Sucrose, Trehalose, Raffinose, Glycine, Sorbitol, Sodium Chloride, Potassium Chloride, Povidone, Polyethylene glycol, or any other inactive ingredient that can be used for parenteral administration and used for lyophilization as bulking agent or filler to form suitable lyophilized cake.

The formulations of the present invention may further comprise a tonicity adjusting agent at 0.5% to 20% w/v of the formulation, preferably 0.9% to 20% w/v. Tonicity adjusting agents suitable for use in pharmaceutical compositions described herein include, but are not limited to, anhydrous or hydrous forms of sodium chloride, dextrose, sucrose, xylitol, fructose, glycerol, sorbitol, mannitol, potassium chloride, mannose other inorganic salts except containing divalent cations. In certain preferable embodiments, the formulations comprise sodium chloride.

The formulations may optionally comprise chelating agent at about 0.5% to 5.0% w/w of the formulation, preferably about 0.5% to 2.0% w/w. Suitable chelating agents which may be used in the present invention include, but not limited to edetate disodium (EDTA); edetate trisodium, edetate tetrasodium; and diethyleneamine pentaacetate or derivatives thereof. In certain preferable embodiments, the formulations comprise disodium edetate.

Optionally, the formulations comprise pH adjusting agent or buffering agent at about 0% to 5% w/w of the formulation, preferably about 0.05% to 2.0%.

Buffers suitable for use in the pharmaceutical compositions described herein include, but are not limited to, pharmaceutically acceptable salts and acids of acetate, glutamate, citrate, tartrate, benzoate, lactate, histidine or other amino acids, gluconate, phosphate, malate, succinate, formate, propionate, and carbonate.

Suitable pH adjusting agents which may be used in the present invention include, but not limited to sodium hydroxide, hydrochloric acid, citric acid, acetic acid, fumaric acid, hydrochloric acid, malic acid, nitric acid, phosphoric acid, propionic acid, sulfuric acid, tartaric acid, or combinations thereof. In certain preferable embodiments, the formulations comprise a pH adjusting agent. In some of those embodiments, the pH adjusting agent is sodium hydroxide and/or hydrochloric acid.

As used herein to describe the formulations of the present invention the term “stable” means a change of not more than about 10% in the concentration of the active agents when measured by HPLC.

Ondansetron is stable at lower pH (about 3.3 to 4.0) and Fosaprepitant at higher pH (about 7 to 9). The present invention overcomes this disparity by provision of a formulation with better stability in the dried state. The combination product of drugs stable at opposite pH in liquid state is addressed by freeze drying a pre-lyophilization solution.

Alkali degradation study of Palonosetron demonstrated that Palonosetron is stable at alkaline pH. As discussed, Fosaprepitant Dimeglumine is stable at higher pH (about 7 to 9). Based on this information, Palonosetron HCl can be used along with Fosaprepitant Dimeglumine in a liquid ready-to-use combination injectable product.⁹ ⁹ http://www.sphinxsai.com/2015/ch_vol8_nol 0/2/(317-337)V8N10CT.pdf at Page 330, 336.

The pre-lyophilization solution is prepared prior to lyophilization to make dried cake or powder. The invention thus comprises a lyophilization solution containing Fosaprepitant Dimeglumine from about 1 mg/mL to 150 mg/mL and Ondansetron HCl from about 0.5 mg/mL to 32 mg/mL for parenteral administration.

The freeze dried product is reconstituted prior to dilution for administration to a patient. The invention thus comprises a reconstituted solution of lyophilized product containing Fosaprepitant Dimeglumine from about 5 mg/mL to 150 mg/mL and Ondansetron HCl from about 0.5 mg/mL to 32 mg/mL for parenteral administration.

The reconstituted solution is diluted prior to administration to a patient. The invention thus comprises a reconstituted and/or diluted solution of lyophilized product containing Fosaprepitant Dimeglumine from about 0.5 mg/mL to 50 mg/mL and Ondansetron HCl from about 0.5 mg/mL to 32 mg/mL for parenteral administration.

In some embodiments, the diluent for reconstitution is water for injection, 0.9% sodium chloride injection, 5% Dextrose, or any other suitable pharmaceutical fluid or combination thereof for parenteral administration.

In certain embodiments, the solution is further diluted at least 5 times before parenteral administration.

In accordance with the present disclosure the 5-HT3 antagonist and the NK-1 receptor antagonist are administered to a patient in a quantity sufficient to treat or prevent the symptoms, and/or underlying etiology associated with emesis in the patient.

The present disclosure will now be described in connection with certain embodiments, which are not intended to be limiting in scope. On the contrary, the present application covers all alternatives, modifications, and equivalents as included within the scope of the claims. Thus, the following will illustrate the practice of the present disclosure, for the purposes of illustration of certain embodiments and is presented to provide what is believed to be a useful and readily understood description of its procedures and conceptual aspects.

### EXAMPLES

### Example 1: Fosaprepitant Ondansetron HCl for Injection, 150 Mg/16 mg Per Vial Lyophilized Combination Product (Batch Size—1.0 L/125 Vials)

| Sr. No. | Ingredient | Qty/mL | Qty/vial | % w/w |
|---|---|---|---|---|
| 1 | Fosaprepitant | 30.663 mg | 245.3 mg | 34.23% |
| 2 | Ondansetron HCl | 2.000 mg | 16.0 mg | 2.23% |
| 3 | Disodium Edetate | 0.675 mg | 5.4 mg | 0.75% |
| 4 | Polysorbate 80 | 9.375 mg | 75.0 mg | 10.46% |
| 5 | Lactose Anhydrous | 46.875 mg | 375.0 mg | 52.32% |
| 6 | Sodium Hydroxide | q.s. to pH | q.s. to pH | q.s. |
| 7 | Hydrochloric acid | q.s. to pH | q.s. to pH | q.s. |
| 8 | Water for Injection | q.s. to 1 mL | q.s. to 8 mL* | — |

*Water for Injection will be removed during lyophilization

Method of Preparation of Solution for Lyophilization:
1. Approx. 70% of required water for injection was collected;
2. Required quantity of Disodium Edetate was added and mixed;
3. Required quantity of Ondansetron HCl was added and mixed;
4. Required quantity of Polysorbate 80 was added and mixed;
5. Required quantity of Fosaprepitant Dimeglumine was added and mixed;
6. Volume of the batch was made up to 95% of batch size;
7. pH of solution was adjusted to about 7 using sodium hydroxide and/or hydrochloric acid;
8. Required quantity of Lactose anhydrous was added and mixed;
9. Volume to batch was made up to batch size using water for injection and filtered using 0.22p filter, 8 mL of filtered solution was filled in glass vial and half stoppered it; and
10. Lyophilized using below lyophilization cycle.

Lyophilization Cycle:

| | | | |
|---|---|---|---|
| Temperature | Time (minutes) | Hold/Ramp | Pressure (mTorr) |

Freezing:

| | | | |
|---|---|---|---|
| 2° C. | 60 | Hold | NA |
| −50° C. | 188 | Ramp | NA |
| −50° C. | 300 | Hold | NA |

Primary drying:

| | | | |
|---|---|---|---|
| −50° C. | 30 | Hold | 150 |
| −26° C. | 270 | Ramp | 150 |
| −26° C. | 3200 | Hold | 150 |
| −26° C. | 3200 | Hold | 150 |
| −5° C. | 600 | Ramp | 150 |
| −5° C. | 600 | Hold | 150 |
| 25° C. | 600 | Ramp | 150 |

Secondary drying:

| | | | |
|---|---|---|---|
| 25° C. | 800 | Hold | 125 |

Observation: After the lyophilization, white cake was obtained with good structure. Reconstitution of the lyophilized vial using 5.0 mL Sodium Chloride Injection resulted in clear, colorless solution. Reconstitution time was about 15 seconds. Additional testing was performed for stability study as below.

TABLE 1
Stability study of Fosaprepitant/Ondansetron HCl for
injection, 150 mg/16 mg per vial lyophilized product

| | | 6 Months | 6 Months |
|---|---|---|---|
| Test | Initial | (2-8° C.) | (25° C./60% RH) |
| Appearance | White to off white | White to off white | White to off white |
| | lyophilized cake | lyophilized cake | lyophilized cake |
| Assay of Fosaprepitant | 101.1% | 103.5% | 98.8% |
| Assay of Ondansetron | 97.9% | 100.2% | 97.0% |
| HCl | | | |
| Water content | 0.20% | 0.29% | 0.24% |
| pH of reconstituted | 6.6 | 6.7 | 6.7 |
| solution | | | |
| Reconstitution time | 19 seconds | 16 seconds | 15 seconds |
| Description of | Clear colorless | Clear colorless | Clear colorless |
| reconstituted solution | solution | solution | solution |

### Example 2: Fosaprepitant/Palonosetron HCl for Injection, 150 Mg/0.25 mg Per Vial Lyophilized Combination Product (Batch Size—500 mL/125 Vials)

| | | | | % w/w |
|---|---|---|---|---|
| Sr. | | | | (after |
| No. | Ingredient | Qty/mL | Qty/vial | lyophilization) |
| 1 | Fosaprepitant | 61.325 mg | 245.3 mg | 34.99% |
| | Dimeglumine | | | |
| 2 | Palonosetron HCl | 0.07 mg | 0.28 mg | 0.04% |
| 3 | Disodium Edetate | 1.35 mg | 5.4 mg | 0.77% |
| 4 | Polysorbate 80 | 18.75 mg | 75 mg | 10.70% |
| 5 | Lactose Anhydrous | 93.75 mg | 375 mg | 53.50% |
| 6 | Sodium Hydroxide | q.s. to pH | q.s. to pH | q.s. |
| 7 | Hydrochloric acid | q.s. to pH | q.s. to pH | q.s. |
| 8 | Water for Injection | q.s. to 1 mL | 4 mL* | — |

*Water for Injection is removed during lyophilization

Method of Preparation of Solution for Lyophilization:
1. Approx. 70% of required water for injection was collected and purged with nitrogen;
2. Required quantity of Lactose anhydrous was added and mixed;
3. Required quantity of Disodium Edetate was added and mixed;
4. Required quantity of Polysorbate 80 was added and mixed;
5. Required quantity of Fosaprepitant Dimeglumine was added and mixed;
6. Volume of the batch was made up to 95% of batch size using nitrogen purged water;
7. pH of solution was adjusted to about 8 using sodium hydroxide and/or hydrochloric acid;
8. Required quantity of Palonosetron HCl was added and mixed;
9. Volume to batch was made up to batch size using nitrogen purged water for injection and filtered using 0.22p filter, 4 mL of filtered solution was filled in glass vial and half stoppered; and
10. Lyophilized using below lyophilization cycle.

Lyophilization Cycle:

| | | | |
|---|---|---|---|
| | | | Pressure |
| Temperature | Time (minutes) | Hold/Ramp | (mTorr) |

Freezing:

| | | | |
|---|---|---|---|
| 2° C. | 60 | Hold | NA |
| −50° C. | 188 | Ramp | NA |
| −50° C. | 300 | Hold | NA |

Primary drying:

| | | | |
|---|---|---|---|
| −50° C. | 30 | Hold | 150 |
| −22° C. | 270 | Ramp | 150 |
| −22° C. | 3200 | Hold | 150 |
| −5° C. | 250 | Ramp | 150 |
| −5° C. | 120 | Hold | 150 |
| 25° C. | 375 | Ramp | 150 |

Secondary drying:

| | | | |
|---|---|---|---|
| 25° C. | 800 | Hold | 150 |

TABLE 2
Stability study of Fosaprepitant/Palonosetron HCl for injection,
150 mg/0.25 mg per vial lyophilized combination product

| | | 6 Months | 6 Months |
|---|---|---|---|
| Test | Initial | (2-8° C.) | (25° C./60% RH) |
| Appearance | Lyophilized | Lyophilized | Lyophilized cake |
| | cake | cake | |
| Assay of | 104.1% | 104.5% | 104.7% |
| Fosaprepitant | | | |
| Assay of | 106.2% | 107.2% | 107.5% |
| Palonosetron HCl | | | |
| Water content | 0.78% | 0.90% | 0.88% |
| pH of reconstituted | 7.7 | 7.7 | 7.7 |
| solution | | | |
| Reconstitution time | <20 seconds | <20 seconds | <20 seconds |
| Description of | Clear colorless | Clear colorless | Clear colorless |
| reconstituted | solution | solution | solution |
| solution | | | |

### Example 3: Fosaprepitant/Palonosetron HCl for Injection, 3 Mg/mL; 5 μg/mL Ready-to-Use Combination Product (Batch Size: 1500 mL)

| Sr. No. | Ingredient | Qty/mL | % w/w |
|---|---|---|---|
| 1 | Fosaprepitant | 4.906 mg | 0.49% |
| | Dimeglumine | | |
| 2 | Palonosetron HCl | 0.0056 mg | 0.00056% |
| 3 | Disodium Edetate | 0.108 mg | 0.0108% |
| 4 | Polysorbate 80 | 1.500 mg | 0.15% |
| 5 | Sodium Chloride | 9.000 mg | 0.9% |
| 6 | Sodium Hydroxide | q.s. to pH | q.s. |
| 7 | Hydrochloric acid | q.s. to pH | q.s. |
| 8 | Water for Injection | q.s. to 1 mL | q.s. to 100% |

Method of Preparation of Solution:
1. Approx. 90% of required water for injection was collected and purged with nitrogen;
2. Required quantity of Sodium chloride was added and mixed;
3. Required quantity of Disodium Edetate was added and mixed;
4. Required quantity of Polysorbate 80 was added and mixed;
5. Required quantity of Fosaprepitant Dimeglumine was added and mixed;
6. Volume of the batch was made up to 95% of batch size using nitrogen purged water for injection;
7. pH of solution was adjusted to about 8 using sodium hydroxide and/or hydrochloric acid;
8. Required quantity of Palonosetron HCl was added and mixed;
9. Volume to batch was made up to batch size and filtered using 0.22p filter; and
10. Filtered solution was filled in glass vial, stoppered with rubber stopper followed by sealing using aluminum seal.

TABLE 2
Stability study results of Fosaprepitant/Palonosetron HCl for
injection, 3 mg/mL; 5 μg/mL ready-to-use combination product

| Test | Initial | 6 Months (2-8° C.) |
|---|---|---|
| Appearance | Clear colorless | Clear colorless |
| | solution | solution |
| Assay of Fosaprepitant | 101.4% | 101.3% |
| Assay of Palonosetron | 102.9% | 103.3% |
| HCl | | |
| pH | 7.7 | 7.7 |

### Example 4: Fosaprepitant/Palonosetron HCl for Injection, 1.5 Mg/mL; 2.5 μg/mL Ready-to-Use Combination Product (Batch Size: 1000 mL)

| Sr. No. | Ingredient | Qty/mL | % w/w |
|---|---|---|---|
| 1 | Fosaprepitant | 2.453 mg | 0.2453% |
| | Dimeglumine | | |
| 2 | Palonosetron HCl | 0.0028 mg | 0.00028% |
| 3 | Disodium Edetate | 0.054 mg | 0.0054% |
| 4 | Polysorbate 80 | 0.75 mg | 0.075% |
| 5 | Sodium Chloride | 0.45 mg | 0.045% |
| 6 | Sodium Hydroxide | q.s. to pH | q.s. |
| 7 | Hydrochloric acid | q.s. to pH | q.s. |
| 8 | Water for Injection | q.s. to 1 mL | q.s. to 100% |

Method of Preparation of Solution for Lyophilization:
1. To the bulk solution of example 3, step 9, equal volume of water for injection was added to dilute the solution to half of its initial concentration; and
2. Final solution was filled in glass vial, stoppered with rubber stopper followed by sealing using aluminum seal.

TABLE 4
Stability study results of Fosaprepitant/Palonosetron HCl for
injection, 1.5 mg/mL; 2.5 μg/mL ready-to-use combination product

| Test | Initial | 6 Months (2-8° C.) |
|---|---|---|
| Appearance | Clear colorless | Clear colorless |
| | solution | solution |
| Assay of Fosaprepitant | 101.8% | 102.1% |
| Assay of Palonosetron | 102.0% | 102.1% |
| HCl | | |
| pH | 7.4 | 7.6 |

While the present teachings have been described above in terms of specific embodiments and examples, it is to be understood that they are not limited to those disclosed embodiments and examples. Many modifications to the embodiments and examples will come to mind to those skilled in the art to which this pertains, and which are intended to be and are covered by both this disclosure and the appended claims. It is intended that the scope of the present teachings should be determined by proper interpretation and construction of the appended claims and their legal equivalents, as understood by those of skill in the art relying upon the disclosure in this specification and the data tables.

1. A liquid sterile pharmaceutical product of Fosaprepitant and a 5-HT3 blocker for parenteral administration comprising:
a container containing a liquid formulation including
Fosaprepitant or a pharmaceutically acceptable salt thereof;
5-HT3 blocker;
surface-active agent at about 0.01% to 20% w/v; and
a pharmaceutically acceptable vehicle.

2. The product of claim 1, wherein the surface-active agent is 0.05% to 15% w/v of the formulation.

3. The product of claim 1, wherein the surface-active agent is Polysorbate 80.

4. The product of claim 1 further comprising at least one of a tonicity agent, pH adjusting agent and chelating agent.

5. The product of claim 1, wherein the 5-HT3 blocker is selected from the group consisting of Ondansetron, Palonosetron, Granisetron, Dolesetron, Tropisetron, and Zatisetron.

6. The product of claim 1, wherein the formulation comprises Fosaprepitant Dimeglumine and Palonosetron Hydrochloride.

7. The product of claim 6, wherein the concentration of Fosaprepitant Dimeglumine is from 0.5 mg/mL to 250 mg/mL and the concentration of Palonosetron Hydrochloride is from 1 μg/mL to 500 μg/mL.

8. A lyophilized sterile pharmaceutical product of Fosaprepitant and 5-HT3 blocker for parenteral administration comprising:
a container containing a lyophilized powder of Fosaprepitant or a pharmaceutically acceptable salt thereof; and a 5-HT3 blocker.

9. The product of claim 8, wherein the 5-HT3 blocker is selected from the group consisting of Ondansetron, Palonosetron, Granisetron, Dolesetron, Tropisetron, and Zatisetron.

10. The product of claim 9, wherein the 5-HT3 blocker is Ondansetron or a salt thereof.

11. The product of claim 10, wherein the Ondansetron is Ondansetron Hydrochloride.

12. The product of claim 8, wherein the 5-HT3 blocker is Palonosetron or a salt thereof.

13. The product of claim 12, wherein the Palonosetron is Palonosetron Hydrochloride.

14. The product of claim 8, wherein the powder is reconstituted with a diluent to contain Fosaprepitant Dimeglumine from about 5 mg/mL to 150 mg/mL and Ondansetron HCl from about 0.5 mg/mL to 32 mg/mL for parenteral administration.

15. The reconstituted product of claim 14, wherein the diluent for reconstitution is water for injection, 0.9% sodium chloride injection, 5% Dextrose, a tonicity adjusting agent or any combination thereof.

16. The reconstituted solution of claim 14, wherein the solution is further diluted at least 5 times before parenteral administration.

17. A pre-lyophilization solution of Fosaprepitant and 5-HT3 blocker comprising about 0.5 mg/mL to 250 mg/mL Fosaprepitant Dimeglumine and at least one of Ondansetron Hydrochloride and Palonosetron Hydrochloride.

18. The pre-lyophilization solution of claim 17 comprising about 0.5 mg/mL to 32 mg/mL Ondansetron Hydrochloride.

19. The pre-lyophilization solution of claim 17 comprising about 0.01 mg/mL to 0.5 mg/mL Palonosetron Hydrochloride.

20. The pre-lyophilization solution of claim 17, wherein the solution contains water, alcohol or a combination thereof as solvent.
